(19) United States
(12) Patent Application Publication
(10) Pub. No.: US 2021/0353275 A1
(43) Pub. Date: Nov. 18, 2021

(54) Hip-Traction Force Monitoring Method And Apparatus

(71) Applicant: Construct Medical Pty Ltd., Woodend (AU)

(72) Inventors: Philip Stuart Esnouf, Burnley (AU); Stuart Kurt Esnouf, Burnley (AU)

(21) Appl. No.: 17/281,116

(22) PCT Filed: Oct. 2, 2019
(86) PCT No.: PCT/AU2019/051070

§ 371 Date: Mar. 29, 2021

(30) Foreign Application Priority Data
Oct. 2, 2018 (AU) 2018903721

(51) Int. Cl. A61B 17/02; A61B 17/56; A61B 1/317; A61B 90/00

(52) U.S. Cl. CPC A61B 17/025; A61B 17/56; A61B 2017/00199; A61B 90/06; A61B 2017/0275; A61B 1/317

(57) ABSTRACT

A system and method for mitigating potential for injurious effects through hip traction forces during a hip arthroscopy procedure or the like utilises a traction force measuring device arranged to measure traction force applied to a patient during the procedure. A digital processor is configured to periodically receive data signals indicative of the measured traction force during the procedure and provide visual indications of applied force and recommended force limits. The processor is configured to generate an alert signal in the event that the measured traction force exceeds a maximum force threshold level that is least partially based on a predetermined body mass (weight) of the patient.

### FIELD

This invention relates to methods and apparatus for use in monitoring force applied during distraction of a hip joint for surgical procedure.

### BACKGROUND

Hip arthroscopy is a procedure performed for both the diagnosis and treatment of many debilitating hip conditions. The surgery involves the introduction of a rigid arthroscope into the hip joint to allow the use of various instruments to be controlled under direct vision. These instruments are introduced through small incisions or portals. Conventional surgical approaches to this joint require substantial surgical dissection of important tissues around the joint and ultimately dislocation of the joint itself, but this can be minimised using an arthroscopic approach.

The hip joint is a ball and socket with a thin layer of fluid separating the joint surfaces. In order to provide a space for the arthroscope to enter the joint, it is necessary to pull the ball (femoral head) away from the socket (acetabulum) by around 10-25 mm. Once this space is created it is the possible to manoeuvre the arthroscope and associated instruments to perform the necessary surgery.

The distraction of the femoral head from the acetabulum requires a traction force to be applied to the patient's femur via the leg. Typically the patient is positioned on an operating table with a distraction device attached e.g. a McCarthy hip distraction unit. The patient may be positioned on their side (lateral position) or on their back (supine). A rigid boot is applied to the patient's foot which is specifically designed to allow a significant amount of traction force to be applied axially along the leg. The patient's pelvis and acetabulum may be held to resist the traction by the placement of a padded bar between the patient's legs—this prevents movement of the ischium and pubic parts of the pelvis.

The traction force can be generated by means of a number of different methods and/or apparatus, an example of which is the aforementioned ‘McCarthy device’ in which a boot containing the patient's foot and lower leg is pulled into tension by a threaded rod mated to a threaded handle and retained against a frame which is fixed to the operating table. When the handle is rotated around the threaded rod, force is applied to the attached boot, the patient's leg, and consequently the femoral head so as to effect movement in an axial direction. The patient's pelvis may be restrained, as mentioned above, whereby sufficient tension force develops to move the femoral head away from the acetabular to allow for the procedure.

In current practice it is common to visualise, via radiographic display, the amount of separation that has occurred as a result of the traction procedure described. Using X-ray imaging, for example, it is possible to see the outline of the ball and socket and judge the separation between them. This separation can be optimised to ensure there is sufficient space to allow for the introduction of the arthroscope and perform the relevant surgery.

The radiographic evidence, however, does not directly correlate to the actual distraction force that is applied to the patient. In practice, initially a larger force (e.g. 400-1000 N) is required to overcome a vacuum generated in the ball-socket joint before separation occurs. Moreover, there may be ligamentous or bone anomalies that require a larger application of force to produce separation in the joint. Once the joint has been instrumented, air enters the joint and the vacuum force is no longer present. At this point in the procedure less force is required to distract the joint or maintain the joint distraction. During the various procedures that are performed, traction may be increased or in many cases removed as a means of changing the position of structures within the joint to optimise surgical conditions.

In most cases the amount of traction applied to the leg is controlled by the surgeon, based on the radiographic evidence, experience from previous procedures and teaching from others. In some cases a tension gauge may be provided in the traction device as a guide.

The use of excessive traction has been shown to cause a number of adverse outcomes or injuries. In most procedures the patient is either not conscious, or has been provided with sufficient pain relief so as not to be able to provide feedback in regard to pain relating to the traction force. Such injuries relate to compression or stretching of tissue in various regions. This includes disruption of ligament and soft tissue around the hip joint, stretching of the sciatic nerve with serious consequences and damage to structures in the knee and foot. The use of the padded bar to restrain the pelvis may cause pressure damage of skin over ischium and pubis including the genitalia and compression of the perineal nerve. Such injuries may be of a temporary or permanent nature.

While excess traction force may result in the problems mentioned, the length of time traction is applied also contributes to these outcomes. It is also recognised that removal of traction or reduction to a lower level, allows many of the pressure sensitive areas e.g. skin and blood vessels to recover and allow for renewed pressure without damage.

There are many instances in surgery where application of potentially injurious force is required to facilitate a procedure. In these cases parameters are carefully monitored and recorded in order to reduce the likelihood of injury. For example, the use of a pneumatic tourniquet commonly used in surgery to a limb, requires careful monitoring of both time and pressure within defined limits in order to prevent injury. Furthermore, in a situation where an injury does occur this monitoring and recording is invaluable to the process of ascertaining whether appropriate care was taken to ensure recognised parameters were met.

### SUMMARY

In one aspect the present invention provides a system for mitigating potential for injurious effects through hip traction forces during a hip arthroscopy procedure or the like, comprising a traction force measuring means arranged to measure traction force applied to a patient during the procedure, a processing means arranged to periodically receive data signals indicative of the measured traction force during the procedure, the processing means being configured to generate an alert signal in the event that the measured traction force exceeds a maximum force threshold level at least partially based on a predetermined body mass (weight) of the patient.

The processing means may be further configured to calculate, during the procedure, a time-force integral approximation and generate an alert signal in the event the time-force integral approximation exceeds a predetermined continuous force threshold. The continuous force threshold may also be at least partially based on the weight of the patient.

In another aspect the present invention provides a method for mitigating potential for injurious effects through hip traction forces during a hip arthroscopy procedure or the like, the method comprising monitoring traction force applied to a patient during the procedure, and generating an alert signal in the event that the measured traction force exceeds a maximum force threshold, or in the event that a time-force integral approximation exceeds a predetermined continuous force threshold, wherein the maximum and continuous force thresholds are at least partially based on a predetermined weight of the patient.

Embodiments of the invention provide a device and associated software that is capable of continuously monitoring the force and traction time during hip arthroscopy. The device is adapted to generate guidelines or suggested limits for such parameters based on individual patient characteristic data in order to provide a surgeon with intraoperative guidance as to safe maximum and continuous traction force, and length of traction time. The information may be communicated both visually and audibly to alert the surgeon in the event that recognised parameters have been exceeded.

Embodiments of the invention include a load cell and associated circuitry capable of being coupled in line with a patient's leg such that force data created by traction is sampled by said load cell. The force data is transmitted using appropriately certified radio frequency (e.g. 2.4 Ghz) to a receiver that interfaces with a compact computer in the form or a tablet or other device with an appropriate screen size to allow easy visualisation by a surgeon at a distance of 1-3 m.

The device and software are capable of collecting and displaying graphic and numeric real-time data along with safety limits calculated based on data input.

The software is comprised of a graphical user interface (GUI), facilitating the above mentioned functions. Patient details are inputted using the GUI including hospital identification number or code, patient's name, date of birth, weight in kilograms and gender. This may also be inputted via a code scanner or hospital data link. Based on these inputs, the software calculates the relevant safety parameters which include but are not limited to: maximum traction force; and continuous traction force which is a function of both traction and time.

Those skilled in the art will appreciate that different embodiments of the this system can be provided, wherein different hardware is used and software that provides this functionality may be in the form of an application suitable for use on a smartphone or other personal handheld device.

### BRIEF DESCRIPTION OF THE DRAWINGS

Further disclosure, objects, advantages and aspects of the present invention may be better understood by those skilled in the relevant art by reference to the following description of preferred embodiments taken in conjunction with the accompanying drawings, which are given by way of illustration only and thus not limitative of the present invention, and in which:

FIG. 1 is a block diagram of a system according to an embodiment of the present invention;

FIG. 2 is a diagrammatic partial perspective view of a patient secured in a hip distraction table for a hip arthroscopy procedure;

FIG. 3 is a diagrammatic sectional view of apparatus for measuring applied traction forces;

FIG. 4 is a diagrammatic view of a processing device having a visual output;

FIG. 5 illustrates an example of a processing device visual display and user interface according to an embodiment of the invention; and

FIG. 6 is a flow chart diagram of a method according to an embodiment of the present invention.

### DETAILED DESCRIPTION

Details of how the invention may be put into practice are provided below, together with specific examples of equipment that may be used to perform various functions. It will be understood, however, that these details and specifics are not to be viewed as restricting the present invention to application with any particular equipment or apparatus.

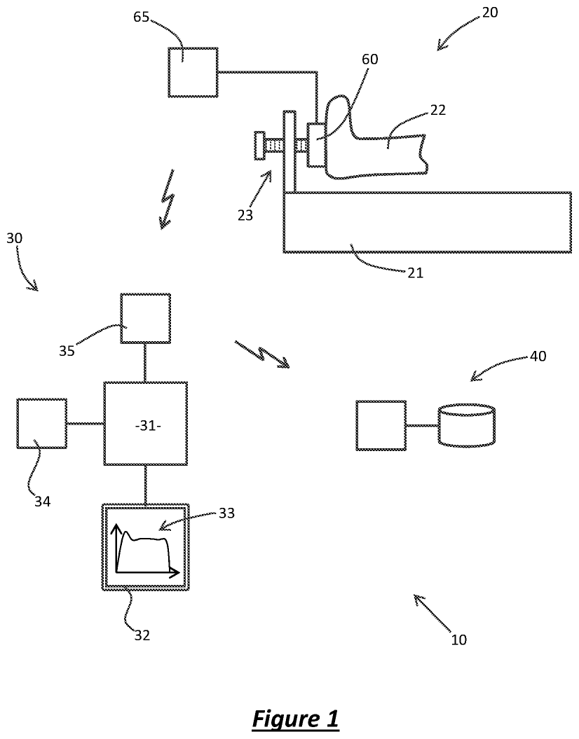

A system 10 according to an embodiment of the present invention is illustrated diagrammatically in FIG. 1, including an arthroscopic procedure apparatus 20, an applied force monitoring apparatus 30 and an optional data storage apparatus 40. The arthroscopic procedure apparatus 20 includes a hip-distraction table 21 equipped with a distraction force application mechanism 23. The distraction force application mechanism 23 may use a screw-threaded mechanism, for example, to displace a rigid boot 22 relative to the table 21, whereby force may be applied to the leg of a patient (not shown) who has a foot fitted within the boot 22 and who is otherwise restrained with respect to the table 21. Interposed between the boot 22 and the mechanism 23 is a force application coupling 60, described in greater detail hereinbelow, that is arranged to detect the amount of tension force applied between the mechanism 23 and the boot 22, representing a measure of the tension force applied to the patient's leg. The force application coupling is connected for communication with a data conditioning and transmitter module 65 capable of wirelessly transmitting data representing the measured tension force.

The force monitoring apparatus 30 includes a data signal receiver 35 adapted to receive signals transmitted by module 65. A programmable computing device 31 is coupled to the receiver 35 to receive the data representing the tension force measured by the coupling 60. The computing device 31 operates according to software instructions, described further below, to analyse the measurement data and provide outputs by way of: visual representations of text data and/or graphical displays 33 shown in real time on a display device such as a touch screen panel 32;

printed text and/or graphical representations generated by a printer 34; and/or data for further analysis and archival purposes wirelessly transmitted or otherwise conveyed to data storage apparatus 40, such as a hospital computer network or the like.

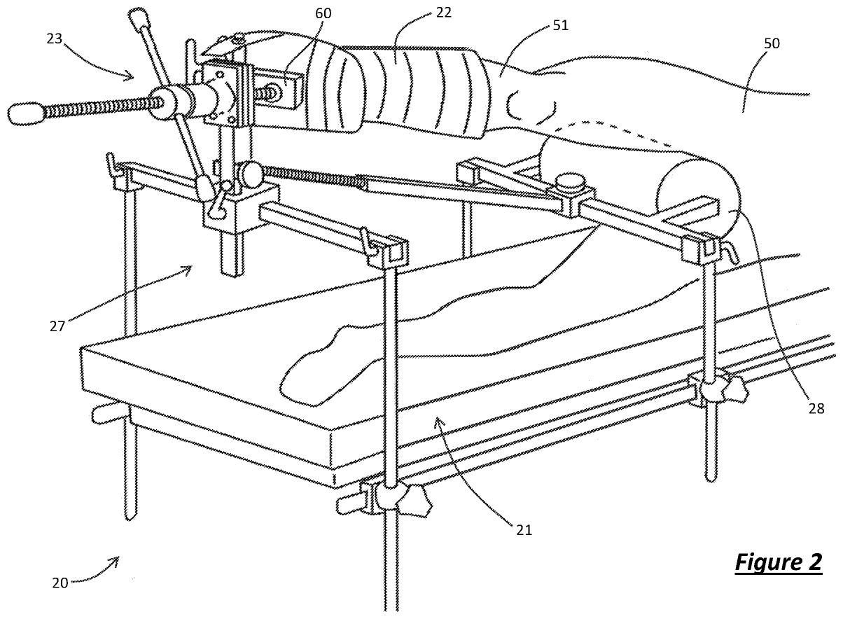

FIG. 2 illustrates an example of the arthroscopic procedure apparatus 20 employing a McCarthy hip-distraction table (available from Innomed Inc. USA, for example). In this diagram, a patient 50 is shown situated on an operating table 21, the foot of the patient's leg 51 being fitted into the rigid boot 22. The boot 22 is connected to distraction force application mechanism 23 by way of coupling 60, and the mechanism 23 is mounted on an adjustable frame 27 that is affixed to the table 21 and has a padded pelvis restrain bar 28. By rotating the screw threaded mechanism 23 the surgeon is able to displace the boot 22 relative to the restrain bar 28, thus applying tension force to the patient's leg 51 for displacing the corresponding hip joint.

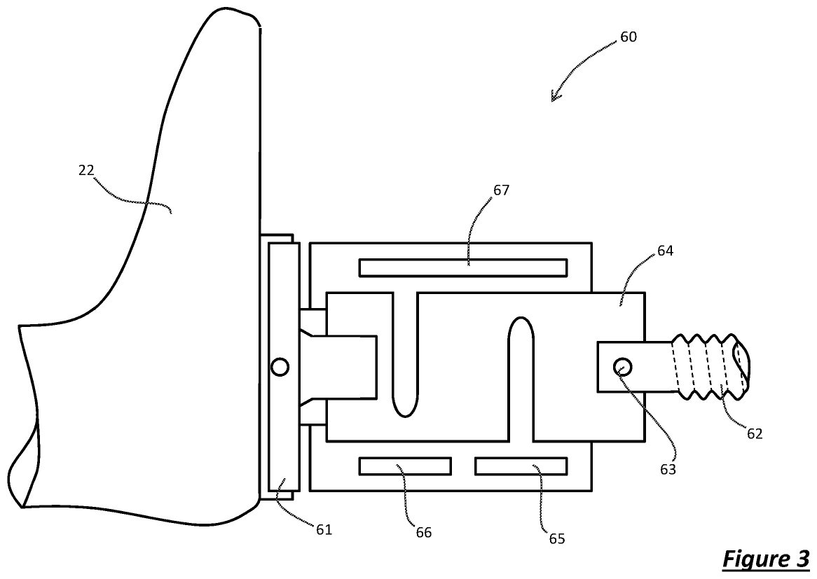

A force application coupling 60 according to embodiments of the invention is operatively connected between the rigid boot 22 and the screw threaded mechanism 23, and is seen in diagrammatic sectional view in FIG. 3. The force application coupling 60 as shown includes:
a load cell 64 (e.g. MT501 Max load 1000KG Millennium Mechatronics N.Z.) connected between a boot mounting 61 and a coupling 63 coupled to a distraction rod 62 of the mechanism 23;
a conditioner and transmitter module 67 (e.g. Mantracourt T24-SA available from Mantracourt Electronics Ltd. UK);
a rechargeable battery 67 and rechargeable battery power supply unit 68 (e.g. Mantracourt module);

In operation, the load cell 64 detects tension forces applied between the boot mounting 61 and the distraction coupling 63 and provides corresponding signals to the module 67. The conditioner and transmitter module 67 filters and digitizes the force measurement signals and transmits the resulting data periodically (e.g. every few seconds or so) to be received by the monitoring apparatus 30.

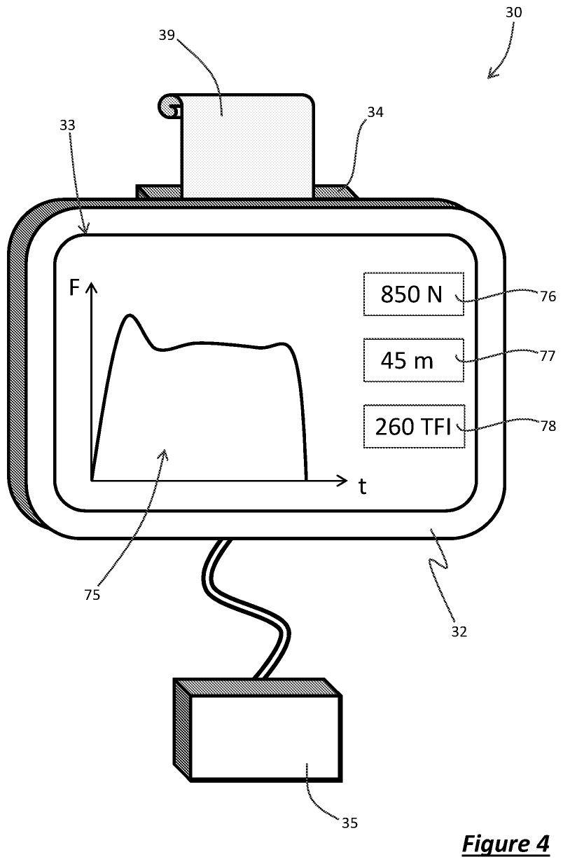

An example of the monitoring apparatus 30 is shown in FIG. 4, comprising a programmable computing device in the form of a touch-screen tablet computer device 32. The tablet computer device 32 may comprise, for example, a Wintec point of sale terminal (e.g. Anypos30 CPU Baytrail Z3735F Ram 2 GB ROM 32 GB available from Qingdao Wintec System Co. Ltd.). The computing device 32 is coupled to receive input from a data receiver 35 (such as a 2.4 GHZ Mantracourt USB data receiver T-24 BSU) which in use receives the periodically transmitted applied force measurement data from the conditioner and transmitter module 67. The computing device 32 may also include or be coupled to a printer 34 for generating a tangible record (print-out 39) of measurement data recorded during a surgical procedure.

The computer device 32 includes a touch-screen display 33 that enables display of text and graphical information (75-78) and also allows the user (e.g. surgeon) to enter data before the surgical procedure for use in the surgical monitoring method as described below. Such data includes, for example, the body mass (weight) of the patient.

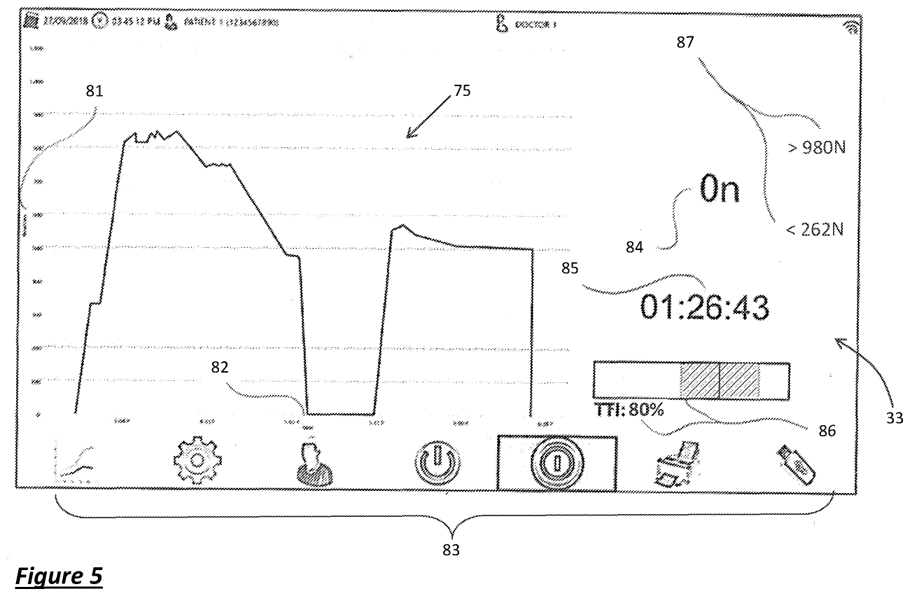

A more detailed example of a display that may be presented on the screen 33 during operation is shown in FIG. 5. This diagram illustrates the means by which a user can interact with the operational software installed on the aforementioned touch-screen tablet in the form of a graphical user interface. Information is conveyed to the user via the following possible graphical elements:
a graph 75 depicting applied distraction force in Newtons on the vertical axis 81 versus time on the horizontal axis 82;
a numerical readout of the instantaneous traction force applied 84;
a numerical readout of the elapsed time of the procedure 85;
a graphical and numerical representation of the time force integral 86;
predetermined upper and lower distraction force thresholds 87; and
touch-screen icon buttons 83 allowing the user to perform actions such as starting and stopping the software, entering patient and procedure data, printing and transferring collected results, etc.

During use of the system according to embodiments of the invention, once an appropriate level of traction is applied to the patient's leg, the unit begins recording and displaying the force data and traction time. This can be initiated manually by pressing the start button on the display. During the operation a number of indicators are displayed including, for example, a graph of force (y-axis) and time (x-axis). An upper traction limit may be displayed as a red line on the graph. Traction force that exceeds this limit generates an audible warning “traction limit exceeded”. The upper limit (in Newtons) is either entered manually by the surgeon (prior to commencement of the procedure) or calculated by the monitor using a formula which is a function of the patient's weight. Similarly, if the traction force is reduced below a lower limit, which can be entered by the surgeon or calculated by the software, an audible sign is generated “traction below limit”. This lower limit serves several purposes: prevention of the software initiating due to the weight of the patients leg; and detection of failures in the traction system such as the patient's foot slipping out of the boot or mechanical failure.

Time based logging (e.g. every 1 second) is utilised to facilitate a calculation of a cumulative total of traction over time. Calculation is performed by first removing trivial data where no traction is applied or force is below the weight force of the patient's leg. A factor of both force and time is then calculated using an area under the graph calculation via a trapezoidal integral approximation. This is represented graphically and numerically as a percentage (0-100) and known as TFI (time force integral). The algorithm is further adapted to allow for reduction of this value when traction is below a predetermined minimum, also based on the patient's weight. Thus the surgeon has the option to adjust procedure time and levels of traction, and even to remove traction for a period of time to remain within parameters that are known to be less likely to cause injury. The data gathered by the unit can be added to a sampling pool and analysed to improve the fidelity of these predictive algorithms.

The device has an inbuilt printer capable of producing a printed record of the data for the surgeon to include in his or her records. Similarly the unit has data output of a standard type so as the data from a procedure can be transferred to a storage means provided for the surgeon for retention or via a secure hospital data link for storage.

Both the force transmitter unit and the receiving tablet device are battery powered and are capable of operating without connection to external power source. The batteries of both units are rechargeable.

### EXPERIMENTAL RESULTS

### Method

A prototype force gauge was developed
Applied to the distraction device
Consecutive patients undergoing hip arthroscopy were monitored
Single surgeon
Patients age, height & weight recorded
Force gauge recorded and plotted
Traction force over time

### Results

337 patients (179M: 169F)
Average age=33.8 years (range: 14-80)
Average weight=75.2 kg (range: 43 to 129)
Average Body Mass Index=24.7 (range: 16.5-38.7)
Average force required=925.2N (range: 402-1868.8)
Average procedure time=32.5 minutes (range: 8-77.5)
Force required correlates with:
   Weight (r=0.455, P<0.001)
   BMI (r=0.240, P<0.001)
   Time (r=0.124, P=0.03)
Age does not have a statistically significant effect
   (r=0.018, P=0.741)

### CONCLUSIONS

Hip arthroscopy performed in the lateral position requires an amount of force that correlates with:
Patient's weight
BMI
This can be calculated by:

F(max(N))=9.81×1.25×M(patient(kg))

If this amount of traction is applied with an operative traction time less than 73 minutes*, the rate of temporary nerve dysfunction can be expected to be less than 2 percent. (*Salas A. and O'Donnell J. Prospective study of nerve injuries associated with hip arthroscopy in the lateral position using the modified portals. J Hip Presery Surg Vol. 3, No. 4, pp. 278-287, the disclosure of which is incorporated herein by reference.)

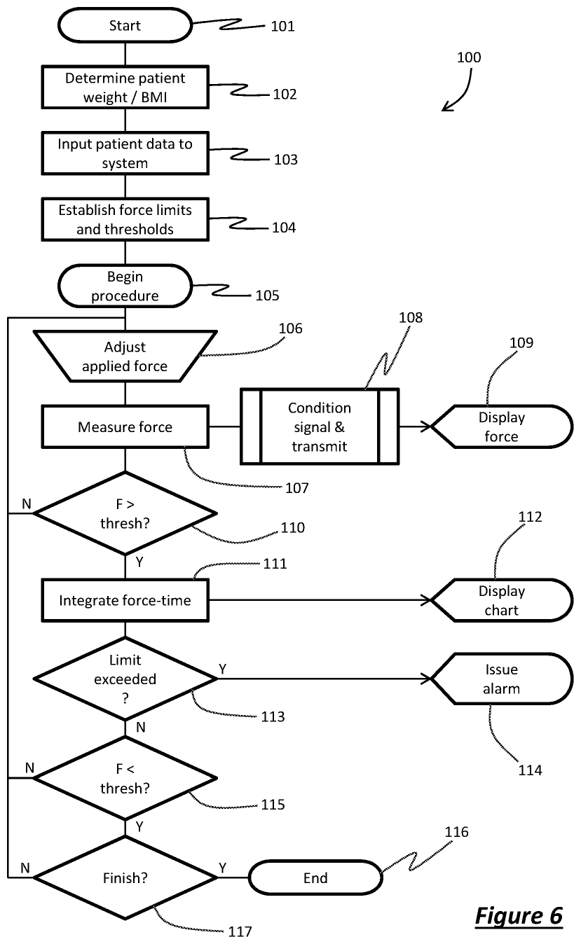

A flow-chart 100 of a method, utilising the system according to embodiments of the present invention, for monitoring applied traction force on a patient during a hip arthroscopic procedure in order to assist the surgeon to avoid use of forces that may be damaging to the patient is shown in FIG. 6. The patient weight and BMI is determined by preoperative measurement (102) and input to the monitoring apparatus computing device (103). The patient weight data enables the computer software to calculate (104) force limits and thresholds for use during the procedure. Once the procedure begins (105) the surgeon adjusts the force applied (106) to the patient's leg using the above described screw thread mechanism, for example. The level of traction force is measured (107) by the load cell in the force application coupling, and corresponding data is transmitted (108) to the monitoring apparatus. The measured force is displayed (109) on the computing device display screen for the surgeon to observe. Once the applied force exceeds a minimum threshold (110) the monitoring apparatus begins recording a force-time integral (111) which is displayed graphically and numerically (112) during the procedure. In the event that the monitoring apparatus determines (113) that the instantaneous applied force or the force-time integral exceeds predetermined limits, based on the measured patient data, an alarm is issued (114) such as an audible signal that can be discerned by the surgeon. This may alert the surgeon to reduce the tension force if necessary. If the measured applied force is determined to be less than a minimum threshold (115), the procedure may be finished (117) or it may be that the surgeon has temporarily relieved the force.

The structure and implementation of embodiments of the invention has been described by way of non-limiting example only, and many additional modifications and variations may be apparent to those skilled in the relevant art without departing from the spirit and scope of the invention described.

Any discussion of documents, devices, acts or knowledge in this specification is included to explain the context of the invention. It should not be taken as an admission that any of the material forms part of the prior art base or common general knowledge in the relevant art in Australia or elsewhere on or before the priority date of the disclosure and claims herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A system for mitigating potential for injurious effects through hip traction forces during a hip arthroscopy procedure or the like, comprising a traction force measuring means arranged to measure traction force applied to a patient during the procedure, a processing means arranged to periodically receive data signals indicative of the measured traction force during the procedure, the processing means being configured to generate an alert signal in the event that the measured traction force exceeds a maximum force threshold level at least partially based on a predetermined body mass of the patient.

2. A system according to claim 1 wherein the processing means is further configured to calculate, during the procedure, a time-force integral approximation and generate an alert signal in the event the time-force integral approximation exceeds a predetermined continuous force threshold.

3. A system according to claim 2 wherein the continuous force threshold is at least partially based on the predetermined body mass of the patient.

4. A method for mitigating potential for injurious effects through hip traction forces during a hip arthroscopy procedure or the like, the method comprising monitoring traction force applied to a patient during the procedure, and generating an alert signal in the event that the measured traction force exceeds a maximum force threshold, or in the event that a time-force integral approximation exceeds a predetermined continuous force threshold, wherein the maximum and continuous force thresholds are at least partially based on a predetermined weight of the patient.
